(19) United States
(12) Patent Application Publication
(10) Pub. No.: US 2024/0188794 A1
(43) Pub. Date: Jun. 13, 2024

(54) BIOLOGICAL-IMAGE GENERATING METHOD AND BIOLOGICAL-IMAGE GENERATING SYSTEM

(71) Applicant: OLYMPUS CORPORATION, Tokyo (JP)

(72) Inventors: Hiromi TAKAHASHI, Tokyo (JP); Koki MORISHITA, Tokyo (JP)

(73) Assignee: OLYMPUS CORPORATION, Tokyo (JP)

(21) Appl. No.: 18/585,612

(22) Filed: Feb. 23, 2024

Related U.S. Application Data
(63) Continuation of application No. PCT/JP2021/032976, filed on Sep. 8, 2021.

(51) Int. Cl. A61B 1/00; A61B 1/06; G06T 7/00

(52) U.S. Cl. CPC A61B 1/000094; A61B 1/00048; A61B 1/0638; G06T 7/00; G06T 2207/10068

(57) ABSTRACT

A biological-image generating method includes: acquiring first and second images of biological tissue respectively irradiated with first and second illumination light; and generating a biological image from the first and second images. The first illumination light has a wavelength which is selected from a range between 500 nm and 600 nm and at which reflectance thereof is not dependent on an oxygen saturation. The second illumination light has a wavelength which is selected from a range between 600 nm and 800 nm and at which reflectance thereof is dependent on the oxygen saturation. The generating includes: calculating an indicator value expressing the oxygen saturation from a first pixel value of each pixel in the first image and a second pixel value of each pixel in the second image; and allocating a display mode corresponding to the indicator value to each pixel of the biological image.

### CROSS-REFERENCE TO RELATED APPLICATIONS

This is a continuation of International Application PCT/JP2021/032976 which is hereby incorporated by reference herein in its entirety.

### TECHNICAL FIELD

The present invention relates to biological-image generating methods and biological-image generating systems.

### BACKGROUND ART

Known technology in the related art involves generating an oxygen saturation image of biological tissue by utilizing a difference between absorption coefficients of oxygenated hemoglobin and reduced hemoglobin (e.g., see Patent Literature 1). An endoscope system according to Patent Literature 1 irradiates the biological tissue with measurement light having a wavelength of 470 nm±10 nm, at which there is a large difference between the absorption coefficients of oxygenated hemoglobin and reduced hemoglobin, acquires an image of the measurement light reflected at the biological tissue, and calculates an oxygen saturation from each pixel value of the acquired image.

### CITATION LIST

### Patent Literature

### PTL 1

Japanese Unexamined Patent Application, Publication No. 2014-76375

### SUMMARY OF INVENTION

A first aspect of the present invention provides a biological-image generating method including: acquiring a first image of biological tissue irradiated with first illumination light; acquiring a second image of the biological tissue irradiated with second illumination light; and generating a biological image from the first image and the second image. The first illumination light has a wavelength which is selected from a range between 500 nm and 600 nm and at which reflectance of the first illumination light is not dependent on an oxygen saturation. The second illumination light has a wavelength which is selected from a range between 600 nm and 800 nm and at which reflectance of the second illumination light is dependent on the oxygen saturation. The generating the biological image includes: calculating an indicator value expressing the oxygen saturation from a first pixel value of each pixel in the first image and a second pixel value of each pixel in the second image; and allocating a display mode corresponding to the indicator value to each pixel of the biological image.

A second aspect of the present invention provides a biological-image generating system including: a light source unit that outputs first illumination light and second illumination light; an imaging unit that includes an image sensor and that acquires a first image of biological tissue irradiated with the first illumination light and a second image of the biological tissue irradiated with the second illumination light; and a processor configured to generate a biological image from the first image and the second image. The first illumination light has a wavelength which is selected from a range between 500 nm and 600 nm and at which reflectance of the first illumination light is not dependent on an oxygen saturation. The second illumination light has a wavelength which is selected from a range between 600 nm and 800 nm and at which reflectance of the second illumination light is dependent on the oxygen saturation. The processor is configured to: calculate an indicator value expressing the oxygen saturation from a first pixel value of each pixel in the first image and a second pixel value of each pixel in the second image; and allocate a display mode corresponding to the indicator value to each pixel of the biological image.

### BRIEF DESCRIPTION OF DRAWINGS

FIG. 1 illustrates the overall configuration of a biological-image generating system according to an embodiment of the present invention.

FIG. 2 illustrates spectra of violet, blue, green, and red light output from a white light source of the biological-image generating system in FIG. 1, and spectral transmission characteristics of a first filter and a second filter.

FIG. 3 is a graph illustrating the relationship between the wavelength and the absorption coefficient of each of oxygenated hemoglobin (HbO2) and reduced hemoglobin (Hb).

FIG. 4 is a graph illustrating the relationship between the blood flow and the reflectance of 584-nm first illumination light with respect to different oxygen saturations.

FIG. 5 is a graph illustrating the relationship between the wavelength and the reflectance of biological tissue with respect to different oxygen saturations.

FIG. 6 is a graph illustrating the relationship between the wavelength and the absorption coefficient of β-carotene.

FIG. 7 is an example of a look-up table in which an indicator value, an oxygen saturation, and a color are associated with one another.

FIG. 8 is a flowchart of a biological-image generating method according to an embodiment of the present invention.

FIG. 9A illustrates an oxygen saturation image of biological tissue generated in accordance with the biological-image generating method according to the embodiment of the present invention before blood vessels are sealed.

FIG. 9B illustrates an oxygen saturation image of the biological tissue generated in accordance with the biological-image generating method according to the embodiment of the present invention after the blood vessels are sealed.

FIG. 10 is a flowchart of a biological-image generating method according to another embodiment of the present invention.

### DESCRIPTION OF EMBODIMENTS

A biological-image generating method and a biological-image generating system according to an embodiment of the present invention will be described below with reference to the drawings.

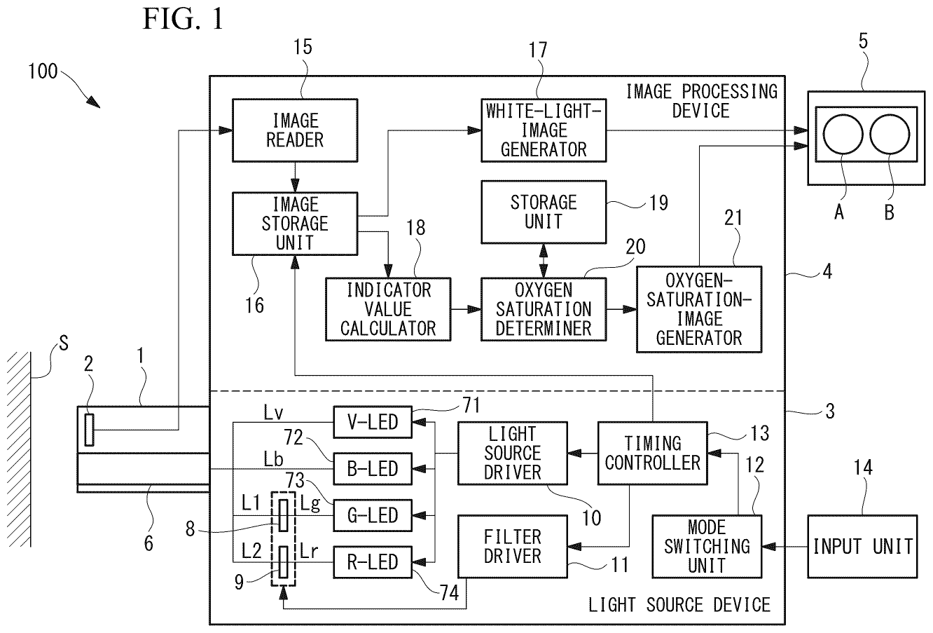

As shown in FIG. 1, a biological-image generating system 100 according to this embodiment is an endoscope system that has a long insertion section 1 to be inserted into a biological organism and that acquires a white light image A and an oxygen saturation image (biological image) B of biological tissue S. The biological-image generating system 100 includes an imaging unit 2 that acquires an image, a light source device (light source unit) 3 and an image processing device (processor) 4 that are connected to the proximal end of the insertion section 1, and a display 5 that is connected to the image processing device 4 and that displays the white light image A and the oxygen saturation image B.

The imaging unit 2 has an imaging element, such as a charge-coupled device (CCD) image sensor or a complementary metal oxide semiconductor (CMOS) image sensor, and is provided at the distal end of the insertion section 1. The imaging unit 2 receives and captures an image of light reflected at the biological tissue S, so as to acquire an image of the biological tissue S. Alternatively, the imaging unit 2 may be provided at the proximal end of the insertion section 1 and capture an image transmitted from the distal end of the insertion section 1 to the imaging unit 2 via a lens system or an image fiber.

The light source device 3 outputs violet light (V light) Lv, blue light (B light) Lb, green light (G (light) Lg, and red light (R light) Lr for acquiring the white light image A. The light source device 3 also outputs first illumination light L1 and second illumination light L2 for acquiring the oxygen saturation image B. The light Lv, Lb, Lg, Lr, L1, and L2 output from the light source device 3 are optically guided to the distal end of the insertion section 1 by a light guide 6 provided in the insertion section 1, and are radiated onto the biological tissue S from the distal end of the insertion section 1.

In detail, the light source device 3 includes four light emitting diodes (LEDs) 71, 72, 73, and 74 that respectively output the V light Lv, the B light Lb, the G light Lg, and the R light Lr, a first filter 8 that generates the first illumination light L1 from the G light Lg, a second filter 9 that generates the second illumination light L2 from the R light Lr, a light source driver 10 that drives the LEDs 71, 72, 73, and 74, a filter driver 11 that drives the filters 8 and 9, a mode switching unit 12 that changes an image mode, and a timing controller 13 that controls the drivers 10 and 11 based on the image mode.

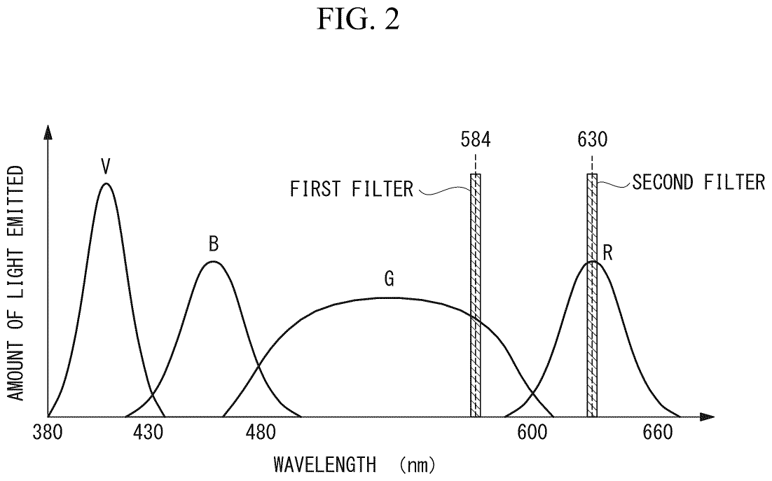

FIG. 2 illustrates spectra of the light Lv, Lb, Lg, and Lr output by the LEDs 71, 72, 73, and 74 and spectral transmission characteristics of the filters 8 and 9. The four LEDs 71, 72, 73, and 74 are white light sources for acquiring the white light image A.

The first filter 8 is a bandpass filter having a center wavelength of 584 nm and is disposed between the G-LED 73 and the light guide 6. The first illumination light L1 having a center wavelength of 584 nm is generated as a result of the G light Lg being transmitted through the first filter 8. The wavelength of the first illumination light L1 is preferably selected from a range between 580 nm and 590 nm. For example, the first illumination light L1 may be light with a single wavelength of 584 nm, or may be light having a wavelength width within a range of 584 nm±5 nm.

The second filter 9 is a bandpass filter having a center wavelength of 630 nm and is disposed between the R-LED 74 and the light guide 6. The second illumination light L2 having a center wavelength of 630 nm is generated as a result of the R light Lr being transmitted through the second filter 9. The wavelength of the second illumination light L2 is preferably selected from a range between 620 nm and 650 nm. For example, the second illumination light L2 may be light with a single wavelength of 630 nm, or may be light having a wavelength width within a range of 630 nm±5 nm.

The filter driver 11 moves the first filter 8 between a position in the optical path of the G light Lg and a position deviated from the optical path of the G light Lg. Furthermore, the filter driver 11 moves the second filter 9 between a position in the optical path of the R light Lr and a position deviated from the optical path of the R light Lr.

Based on an input from a user, the mode switching unit 12 switches between a white-light-image mode for acquiring the white light image A and an oxygen-saturation-image mode for acquiring the oxygen saturation image B. For example, the mode switching unit 12 is connected to an input unit 14 having input devices, such as a mouse, a keyboard, and a touchscreen. The user can use the input unit 14 to switch between the white-light-image mode and the oxygen-saturation-image mode at an arbitrary timing.

In the white-light-image mode, the timing controller 13 controls the filter driver 11 to set the filters 8 and 9 to the positions deviated from the optical paths, and controls the light source driver 10 to sequentially turn on the four LEDs 71, 72, 73, and 74 in synchronization with the imaging process by the imaging unit 2. Accordingly, the V light Lv, the B light Lb, the G light Lg, and the R light Lr are sequentially radiated onto the biological tissue S in synchronization with the imaging timing of the imaging unit 2, so that a V image, a B image, a G image, and an R image are sequentially acquired by the imaging unit 2. The V image, the B image, the G image, and the R image are images of the biological tissue S irradiated with the V light Lv, the B light Lb, the G light Lg, and the R light Lr, respectively.

In the oxygen-saturation-image mode, the timing controller 13 controls the filter driver 11 to set the filters 8 and 9 to the positions in the optical paths, and controls the light source driver 10 to sequentially turn on the G-LED 73 and the R-LED 74 in synchronization with the imaging process by the imaging unit 2. Accordingly, the first illumination light L1 and the second illumination light L2 are sequentially radiated onto the biological tissue S in synchronization with the imaging timing of the imaging unit 2, so that a first image and a second image are sequentially acquired by the imaging unit 2. The first image is an image of the biological tissue S irradiated with the first illumination light L1, and the second image is an image of the biological tissue S irradiated with the second illumination light L2.

The image processing device 4 includes an image reader 15 that reads an image from the imaging unit 2, an image storage unit 16 that temporarily stores the read image, a white-light-image generator 17 that generates the white light image A from the V image, the B image, the G image, and the R image, an indicator value calculator 18 that calculates an indicator value expressing an oxygen saturation from the first image and the second image, a storage unit 19 that stores a look-up table (LUT) in which the indicator value and the oxygen saturation are associated with each other, an oxygen saturation determiner 20 that determines the oxygen saturation from the indicator value based on the LUT, and an oxygen-saturation-image generator 21 that generates the oxygen saturation image B.

The image processing device 4 includes at least one processor, such as a central processing unit, a memory, and a storage unit. The storage unit is a nonvolatile storage medium, such as a read-only memory (ROM) or a hard disk drive, and stores a biological-image generating program. The processor executes a process in accordance with the biological-image generating program loaded in the memory, so as to realize functions, to be described later, of the units 15, 17, 18, 20, and 21. The functions of the image processing device 4 may partially be realized by a dedicated circuit.

The image reader 15 successively reads the images acquired by the imaging unit 2 from the imaging unit 2 and stores the images in the image storage unit 16.

The image storage unit 16 transfers the images to the white-light-image generator 17 or the indicator value calculator 18 based on a signal from the timing controller 13. Specifically, in the white-light-image mode, the image storage unit 16 transfers the V image, the B image, the G image, and the R image to the white-light-image generator 17. In the oxygen-saturation-image mode, the image storage unit 16 transfers the first image and the second image to the indicator value calculator 18.

The white-light-image generator 17 combines the V image, the B image, the G image, and the R image so as to generate the white light image A.

The indicator value calculator 18 calculates an indicator value I of each pixel in the oxygen saturation image B from a first pixel value P1 of each pixel in the first image and a second pixel value P2 of each pixel in the second image. The indicator value I is a difference between the pixel values P1 and P2 at the same position in the first image and the second image. In detail, the indicator value calculator 18 calculates a difference P2−P1 as the indicator value I by subtracting the first pixel value P1 from the second pixel value P2.

The characteristics of the first illumination light L1 having the center wavelength of 584 nm and the second illumination light L2 having the center wavelength of 630 nm will now be described.

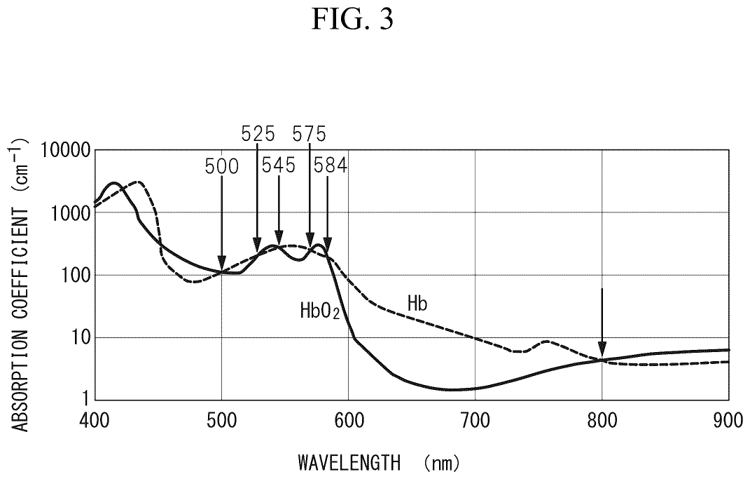

FIG. 3 illustrates the relationship between the absorption coefficient and the wavelength of each of oxygenated hemoglobin (HbO₂) and reduced hemoglobin (Hb).

The 584-nm first illumination light L1 is characterized in that it is affected by the blood flow in the biological tissue S but is not affected by the oxygen saturation in the biological tissue S.

In detail, HbO₂ and Hb each have absorption at 584 nm, and the absorption coefficient of HbO₂ and the absorption coefficient of Hb are equal to each other. This implies that the reflectance of the first illumination light L1 in the biological tissue S is dependent on the blood flow but is not dependent on the oxygen saturation. Therefore, the pixel value P1 of the first image can be used as an indicator for the blood flow.

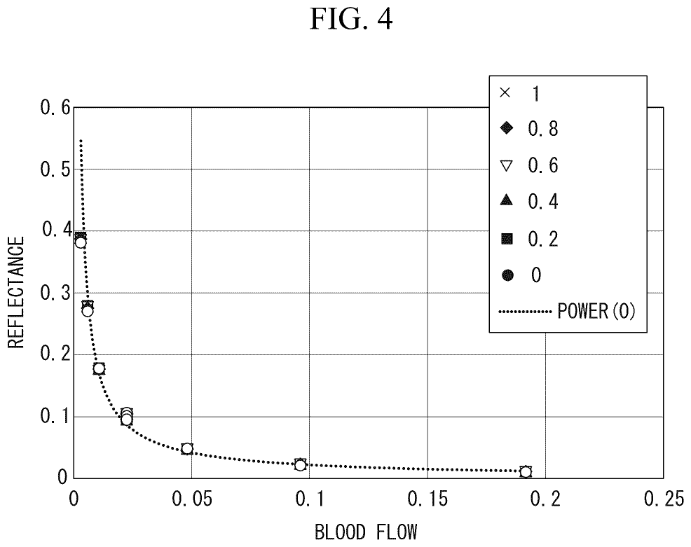

FIG. 4 illustrates simulation results of changes in the reflectance of 584-nm light in the biological tissue S relative to changes in the blood flow when the oxygen saturations are 0%, 20%, 40%, 60%, 80%, and 100%. As illustrated in FIG. 4, the changes in the reflectance match at all of the oxygen saturations 0%, 20%, 40%, 60%, 80%, and 100%, and the reflectance decreases with decreasing blood flow.

The 630-nm second illumination light L2 is characterized in that it is affected by both the blood flow and the oxygen saturation.

In detail, HbO₂ and Hb each have absorption at 630 nm, and the absorption coefficient of HbO₂ and the absorption coefficient of Hb are different from each other. This implies that the reflectance of the second illumination light L2 in the biological tissue S is dependent on the blood flow, and is also dependent on the oxygen saturation. Therefore, the pixel value P2 of the second image can be used as an indicator for the oxygen saturation if the effect of the blood flow can be eliminated.

By using the 630-nm second illumination light L2, information about the oxygen saturation in the biological tissue S can be obtained with high accuracy.

In detail, as illustrated in FIG. 3, the absorption by HbO₂ and Hb tends to decrease with increasing wavelength. 630 nm is a wavelength at which the difference between the absorption coefficient of HbO₂ and the absorption coefficient of Hb becomes the maximum in a wavelength range of 600 nm or higher in which the absorption by HbO₂ and Hb is small. Therefore, by using the 630-nm second illumination light L2, the amount of reflection light of the second illumination light L2 to be image-captured by the imaging unit 2 increases, and a change in the amount of reflection light of the second illumination light L2 caused by a difference in the oxygen saturation becomes the maximum. As a result, an accurate oxygen saturation in the biological tissue S can be estimated from the second pixel value P2.

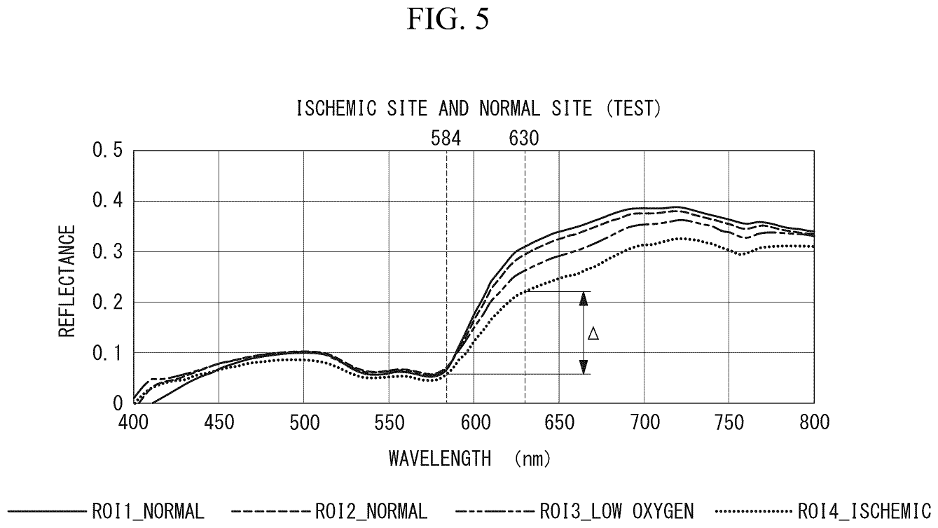

FIG. 5 illustrates test results obtained by measuring changes in the reflectance of illumination light in the biological tissue S relative to changes in the wavelength of the illumination light with respect to a plurality of oxygen saturations. As illustrated in FIG. 5, a change (gradient) Δ in the reflectance between 584 nm and 630 nm is correlated with the oxygen saturation. The gradient Δ increases with increasing oxygen saturation. The indicator value I corresponds to the gradient Δ between 584 nm and 630 nm. Therefore, the oxygen saturation can be estimated from the indicator value I. Moreover, a low oxygen region where the oxygen saturation is lower than normal can be sorted into two abnormality regions, namely, an oxygen lacking region at a caution level where the oxygen saturation is lower than normal and but is not ischemic and an ischemic region at a dangerous level. Threshold values for the oxygen lacking region and the ischemic region are preliminarily set in accordance with the test value Δ. As will be described later, the threshold values are preferably provided in a stepwise fashion.

Furthermore, the 584-nm first illumination light L1 and the 630-nm second illumination light L2 are characterized in that they are not affected by β-carotene contained in fat tissue.

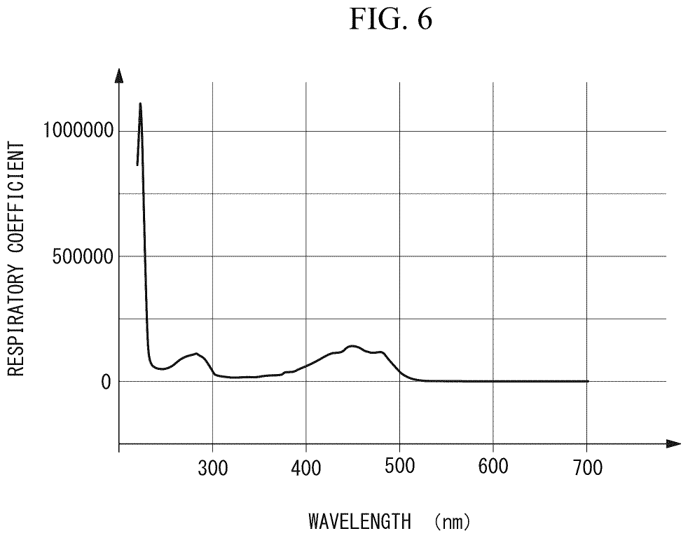

FIG. 6 illustrates the relationship between the wavelength and the absorption coefficient of β-carotene. β-carotene has absorption in a wavelength range lower than 500 nm, but hardly has absorption in a wavelength range higher than or equal to 500 nm. The surface of the biological tissue S is sometimes covered with fat tissue, and the thickness of fat tissue varies from individual to individual and from site to site. The reflectance of each of the illumination light L1 and L2 in the biological tissue S, that is, each of the pixel values P1 and P2 of the first image and the second image, is not dependent on the presence or absence of fat tissue and on the thickness of fat tissue, so that an indicator value I not affected by fat tissue is obtained.

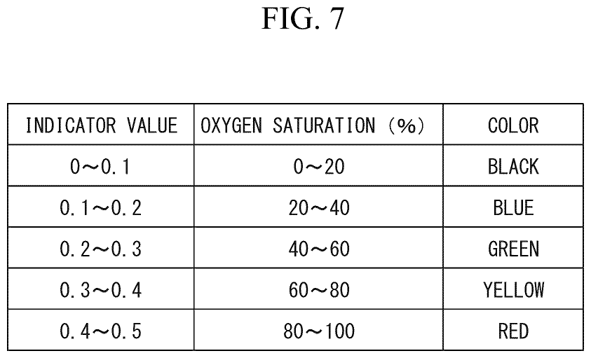

The storage unit 19 is a nonvolatile storage medium, such as a ROM or a hard disk drive. FIG. 7 illustrates an example of an LUT stored in the storage unit 19. In the LUT, an indicator value, an oxygen saturation, and a color are associated with one another. The oxygen saturation is divided into a plurality of levels in accordance with the magnitude, and a range of the indicator value corresponding to each level is set. The oxygen saturation increases with increasing indicator value. In the case of the example in FIG. 7, the oxygen saturation is divided into five levels in 20% increments. Such an LUT is created based on the relationship between the indicator value and the oxygen saturation experimentally obtained in advance.

The oxygen saturation determiner 20 refers to the LUT stored in the storage unit 19 and determines an oxygen saturation from each indicator value based on the LUT. The oxygen saturation determiner 20 determines the level of an oxygen saturation corresponding to an indicator value for each pixel.

The oxygen-saturation-image generator 21 refers to the LUT and allocates a color according to the oxygen saturation of each pixel to the pixel based on the LUT, so as to generate the oxygen saturation image B. The color is any one of the hue, lightness, and chroma, or a combination thereof. In the case of the example in FIG. 7, the pixels of the oxygen saturation image B are each displayed with a color of any one of black, blue, green, yellow, and red colors in accordance with the level of the oxygen saturation.

The operation of the biological-image generating system 100 will now be described.

The biological-image generating system 100 generates the white light image A or the oxygen saturation image B in accordance with the image mode selected by the mode switching unit 12.

In the white-light-image mode, the timing controller 13 controls the filter driver 11 to set the filters 8 and 9 to the positions deviated from the optical paths, and controls the light source driver 10 to cause the LEDs 71, 72, 73, and 74 to sequentially emit light in synchronization with the imaging timing of the imaging unit 2. Accordingly, V light, B light, G light, and R light are sequentially radiated onto the biological tissue S from the distal end of the insertion section 1, and a V image, a B image, a G image, and an R image of the biological tissue S are sequentially acquired by the imaging unit 2.

The V image, the B image, the G image, and the R image are sequentially read from the imaging unit 2 to the image processing device 4 via the image reader 15, are temporarily stored in the image storage unit 16, and are subsequently processed by the white-light-image generator 17. The white-light-image generator 17 combines the V image, the B image, the G image, and the R image so as to generate a white light image. The white light image is transmitted from the image processing device 4 to the display 5, and is displayed on the display 5.

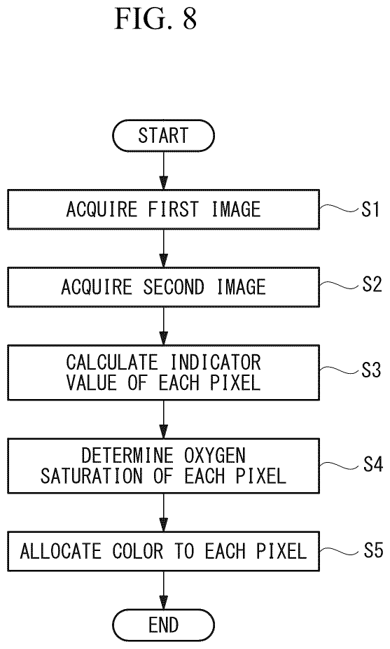

In the oxygen-saturation-image mode, the biological-image generating system 100 executes a biological-image generating method illustrated in FIG. 8 to generate the oxygen saturation image B.

The biological-image generating method includes step S1 for acquiring a first image of the biological tissue S irradiated with the first illumination light L1, step S2 for acquiring a second image of the biological tissue S irradiated with the second illumination light L2, and step S3 to step S5 for generating an oxygen saturation image from the first image and the second image.

In step S1, the timing controller 13 controls the filter driver 11 to set the first filter 8 to the position in the optical path, and controls the light source driver 10 to cause the G-LED 73 to emit light in synchronization with the imaging timing of the imaging unit 2. Accordingly, the first illumination light L1 is radiated onto the biological tissue S from the distal end of the insertion section 1, and the first image of the biological tissue S is acquired by the imaging unit 2.

In step S2, the timing controller 13 controls the filter driver 11 to set the second filter 9 to the position in the optical path, and controls the light source driver 10 to cause the R-LED 74 to emit light in synchronization with the imaging timing of the imaging unit 2. Accordingly, the second illumination light L2 is radiated onto the biological tissue S from the distal end of the insertion section 1, and the second image of the biological tissue S is acquired by the imaging unit 2.

The first image and the second image are sequentially read from the imaging unit 2 to the image processing device 4 via the image reader 15, are temporarily stored in the image storage unit 16, and are subsequently used in step S3 to step S5 for generating an oxygen saturation image.

The steps for generating an oxygen saturation image include step S3 for calculating an indicator value I indicating an oxygen saturation, step S4 for determining the oxygen saturation from the indicator value I, and step S5 for allocating a color to each pixel of the oxygen saturation image B based on the oxygen saturation.

In step S3, the indicator value calculator 18 subtracts a pixel value P1 of each pixel of the first image from a pixel value P2 of each pixel of the second image, so as to calculate a difference P2−P1 as the indicator value I.

In step S4, the oxygen saturation determiner 20 determines the oxygen saturation of each pixel from the indicator value I of each pixel based on the LUT.

In step S5, the oxygen-saturation-image generator 21 allocates a color corresponding to the indicator value I to each pixel of the oxygen saturation image B based on the LUT. Accordingly, a heat map in which the oxygen saturation at each position of the biological tissue S is expressed by color is generated as the oxygen saturation image B.

The oxygen saturation image B is transmitted from the image processing device 4 to the display 5, and is displayed on the display 5. The user can intuitively check the oxygen saturation at each position in the biological tissue S based on the color at each position in the oxygen saturation image B. In order to allow the user to readily compare the oxygen saturation image B and the white light image A with each other, the white light image A and the oxygen saturation image B may be displayed side-by-side on the display 5. Alternatively, the oxygen saturation image B superposed on the white light image A may be displayed on the display 5.

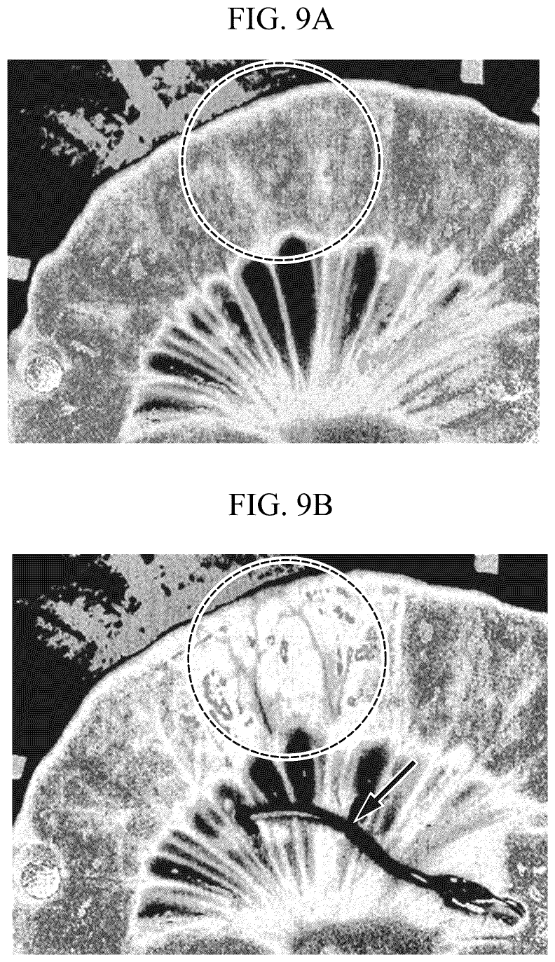

FIGS. 9A and 9B each illustrate the oxygen saturation image generated in accordance with the biological-image generating method according to this embodiment. By sealing some blood vessels with a device (see an arrow in FIG. 9B), a partial region (i.e., a region surrounded by a circle) of the biological tissue S where fat tissue is present is set in a low oxygen state. FIG. 9A illustrates the oxygen saturation image before the blood vessels are sealed, and FIG. 9B illustrates the oxygen saturation image after the blood vessels are sealed. In the region set in the low oxygen state within the oxygen saturation image in FIG. 9B, the color has changed, as compared with the oxygen saturation image in FIG. 9A. It is apparent that the oxygen saturation of the biological tissue S is clearly captured without being affected by the fat tissue.

Accordingly, in this embodiment, the first image is acquired by using the first illumination light L1 that is not affected by the oxygen saturation, and the second image is acquired by using the second illumination light L2 that is affected by the oxygen saturation.

In addition to the oxygen saturation, the second pixel value P2 of the second image includes information about noise derived from the biological tissue S, such as the blood flow and a pigment other than hemoglobin Hb and hemoglobin HbO₂. On the other hand, the first pixel value P1 of the first image does not include information about the oxygen saturation, and includes information about noise derived from the biological tissue S. Therefore, by using the first pixel value P1, a component derived from noise can be removed from the second pixel value P2, and the indicator value I indicating the oxygen saturation can be obtained. Based on such an indicator value I, an oxygen saturation image accurately expressing the oxygen saturation of the biological tissue S can be generated.

Furthermore, since the first illumination light L1 and the second illumination light L2 both have a wavelength greater than or equal to 500 nm at which β-carotene does not have absorption, the first pixel value P1 and the second pixel value P2 are not affected by β-carotene. Therefore, an oxygen saturation image that accurately expresses the oxygen saturation of the biological tissue S can be generated regardless of the presence or absence of fat tissue in the biological tissue S and the thickness of fat tissue.

Furthermore, with the use of the first illumination light L1 having the center wavelength of 584 nm, the accuracy of the indicator value I can be enhanced.

Specifically, as illustrated in FIG. 3, in addition to 584 nm, the visible range has a plurality of wavelengths (e.g., 500 nm, 525 nm, 545 nm, and 575 nm) at which the absorption coefficient of HbO₂ and the absorption coefficient of Hb are equal to each other. The light with these wavelengths is not affected by the oxygen saturation similarly to the 584-nm first illumination light L1.

Among the plurality of wavelengths (e.g., 500 nm, 525 nm, 545 nm, 575 nm, and 584 nm) at which the absorption coefficient of HbO₂ and the absorption coefficient of Hb are equal to each other, the gradient Δ between 584 nm and 630 nm becomes the maximum. Therefore, by using 584 nm, a change in the indicator value I relative to a difference in oxygen saturations is maximized, so that the indicator value I and the oxygen saturation can be calculated with higher accuracy. Furthermore, among 500 nm, 525 nm, 545 nm, 575 nm, and 584 nm, 584 nm is the wavelength closest to 630 nm, and the effect of noise received by the first illumination light L1 is equal to or substantially equal to the effect of noise received by the second illumination light L2. Therefore, by using the first pixel value P1, the effect of noise can be eliminated with high accuracy from the second pixel value P2, so that the indicator value I can be calculated with higher accuracy.

Furthermore, by using the second illumination light L2 having the center wavelength of 630 nm, the accuracy of the indicator value I can be enhanced.

Specifically, in addition to 630 nm, the visible range has a wavelength at which the difference between the absorption coefficient of HbO₂ and the absorption coefficient of Hb is large. For example, the difference between the absorption coefficient of HbO₂ and the absorption coefficient of Hb is large at around 470 nm. However, since there is large absorption by HbO₂ and Hb at around 470 nm, the amount of reflection light decreases, thus making it difficult to achieve sufficient accuracy of the indicator value I. For example, the amount of reflection light of the illumination light changes due to variations in observation conditions, such as different observation distances from the distal end of the insertion section 1 to the biological tissue S, thus making it difficult to distinguish between a change in the amount of reflection light caused by a difference in oxygen saturations and a change in the amount of reflection light caused by other factors.

On the other hand, since the absorption by HbO₂ and Hb is small at 630 nm, the amount of reflection light increases. Therefore, high accuracy of the indicator value I can be readily achieved.

In the above embodiment, the oxygen-saturation-image generator 21 allocates the color according to the oxygen saturation to each pixel of the oxygen saturation image. However, the display mode of the oxygen saturation image is not limited to this and is changeable, where appropriate.

In one modification, the oxygen-saturation-image generator 21 may allocate the density (lightness) or chroma according to the oxygen saturation to each pixel in place of the color. The density or the chroma may increase with increasing oxygen saturation. In another modification, the oxygen-saturation-image generator 21 may vary the density or chroma in accordance with the oxygen saturation in place of the color. For example, a difference in oxygen saturations at the same level may be expressed using the density or chroma.

Alternatively, the oxygen-saturation-image generator 21 may allocate a hatched area according to each level of oxygen saturation to a region of the level.

In the above embodiment, the image processing device 4 may further determine a low oxygen region where the oxygen saturation is lower than or equal to a predetermined threshold value.

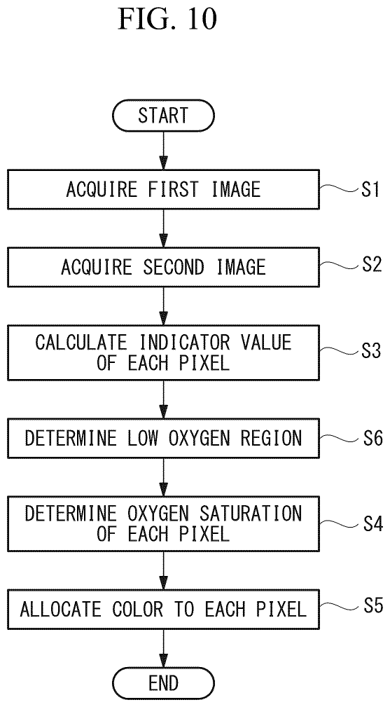

For example, as illustrated in FIG. 10, it may be determined that a pixel where the indicator value I is lower than equal to the threshold value is a low oxygen region (step S6). Alternatively, after the oxygen saturation of each pixel is determined (step S4), it may be determined that a pixel where the oxygen saturation is at a level lower than or equal to a predetermined level is a low oxygen region.

In this case, the oxygen-saturation-image generator 21 may allocate a display mode to each pixel such that at least the low oxygen region is identifiable. For example, in order to display each low oxygen region in a highlighted fashion in the oxygen saturation image B, the oxygen-saturation-image generator 21 may allocate a color different from those of other regions to the low oxygen region.

For a surgeon performing an operation on a biological organism, it is important to recognize a low oxygen region, such as an ischemic region, where the oxygen saturation is low. By allocating a display mode different from those of other regions to each low oxygen region, the surgeon can readily recognize the low oxygen region in the biological tissue S.

In order for the surgeon to be able to recognize a low oxygen region at a glance, it is preferable that the low oxygen region be displayed in a conspicuous color (hue, lightness, chroma) relative to other regions. Moreover, in order for the surgeon to recognize the oxygen saturation in each low oxygen region more specifically, a display mode (e.g., density or chroma) according to the magnitude of the indicator value I may be allocated to each pixel in the low oxygen region.

Instead of or in addition to varying the display mode of other regions from the display mode of each low oxygen region, the oxygen-saturation-image generator 21 may add a mark to the low oxygen region in the oxygen saturation image B. For example, a line surrounding the low oxygen region may be displayed on the oxygen saturation image B. Accordingly, the surgeon can recognize the low oxygen region more readily.

As an alternative to the above embodiment in which the center wavelength of the first illumination light L1 is 584 nm, the center wavelength may be another wavelength at which the absorption coefficient of HbO₂ and the absorption coefficient of Hb are equal to each other.

As mentioned above, the wavelength of the first illumination light L1 is preferably at around the wavelength of the second illumination light L2. Furthermore, the wavelength of the first illumination light L1 is preferably greater than or equal to 500 nm. Therefore, the wavelength of the first illumination light L1 is preferably selected from a range between 500 nm and 600 nm, and is preferably selected from a wavelength at around 500 nm, a wavelength at around 525 nm, a wavelength at around 545 nm, a wavelength at around 575 nm, and a wavelength at around 584 nm.

Light with wavelengths of 500 nm, 525 nm, 545 nm, and 575 nm is also not affected by the oxygen saturation. Therefore, even when the first illumination light L1 used has the center wavelength at around 500 nm, 525 nm, 545 nm, or 575 nm, the indicator value I expressing the oxygen saturation can be calculated, and an oxygen saturation image accurately expressing the oxygen saturation of the biological tissue S can be generated.

As an alternative to the above embodiment in which the center wavelength of the second illumination light L2 is 630 nm, the center wavelength may be another wavelength at which the absorption coefficient of HbO₂ and the absorption coefficient of Hb are not equal to each other.

As mentioned above, the wavelength of the second illumination light L2 is preferably greater than or equal to 600 nm at which absorption by HbO2 and Hb is small. Therefore, the wavelength of the second illumination light L2 is preferably selected from a range between 600 nm and 800 nm, and is more preferably selected from a range between 620 nm and 650 nm in which the difference particularly increases.

As an alternative to the above embodiment in which the indicator value calculator 18 calculates the difference P2−P1 between the pixel values P1 and P2 as the indicator value I, the indicator value calculator 18 may calculate a ratio between the pixel values P1 and P2.

For example, the indicator value I may be a ratio P1/P2 of the first pixel value P1 to the second pixel value P2. Similar to the difference P2−P1, the ratio P1/P2 can also be used as the indicator value I expressing the oxygen saturation.

In the above embodiment, a plurality of blood-flow LUTs that are different from each other may be stored in the storage unit 19.

The blood flow may vary depending on the type or the site of the biological tissue S. The amount of reflection light of each of the illumination light L1 and L2 is affected by the blood flow. In a case where the biological tissue S has a high blood flow, the pixel values P1 and P2 are smaller. Therefore, in a case where the oxygen saturation of biological tissue S with a high blood flow and the oxygen saturation of biological tissue S with a low blood flow are determined by using the same LUT, the accuracy of the oxygen saturation may decrease.

Therefore, the LUT used for determining the oxygen saturation may be selected from the plurality of LUTs in accordance with the blood flow. For example, an LUT may be prepared and stored for every type of biological tissue S, such as the large intestine, the small intestine, the stomach, and the esophagus.

As mentioned above, since the first pixel value P1 is an indicator for the blood flow, an LUT to be used for determining the oxygen saturation may be selected based on the first pixel value P1. For example, if the first pixel value P1 is within a predetermined range, a first standard-blood-flow LUT may be used. If the first pixel value P1 is larger than the predetermined range, a second high-blood-flow LUT may be used.

As an alternative to the above embodiment in which colors corresponding to indicator values I are allocated to all of the pixels in the oxygen saturation image B, a color may be allocated only to a target region within the oxygen saturation image B, so that an oxygen saturation image B displaying only the target region may be generated.

For example, in a case where the observation target is the large intestine, the target region is a region excluding a region other than the large intestine. Since an excessively bright region and an excessively dark region have a high possibility of being noise regions, the target region may be a region further excluding the excessively bright region and the excessively dark region. For example, the target region is extracted based on the indicator value I, the pixel value P1 of the first image, or the color of the white light image.

The ranges of the indicator value I and the oxygen saturation in the target region of the biological tissue S are already known from a preliminarily-performed test. By generating an oxygen saturation image B including the target region alone, the oxygen saturation in the target region can be displayed with higher resolution.

For example, in the case of the LUT in FIG. 7, the ranges of indicator values between 0 and 0.5 are displayed in five levels using black, blue, green, yellow, and red colors. In contrast, if the oxygen saturation image B includes the target region alone and the indicator value I in the target region is between 0.1 and 0.3, the range between 0.1 and 0.3 can be displayed in five levels using black, blue, green, yellow, and red colors.

In the above embodiment, the first illumination light L1 may have a center wavelength at around 820 nm.

The absorption coefficient of HbO₂ and the absorption coefficient of Hb are also equal to each other at around 820 nm. Therefore, even when the first illumination light L1 used has the center wavelength at around 820 nm, the indicator value I expressing the oxygen saturation can be calculated, and an oxygen saturation image accurately expressing the oxygen saturation of the biological tissue S can be generated.

Furthermore, Hb and HbO₂ have less absorption at 820 nm than at 584 nm. Therefore, with the use of the first illumination light L1 with the wavelength at around 820 nm, a first pixel value P1 with a reduced effect of absorption by Hb and HbO₂ can be obtained. Furthermore, the effect of scattering caused by fat is reduced, so that the amount of reflection light of the first illumination light L1 increases, whereby the accuracy of the first pixel value P1 is stable.

In this case, a light source that outputs light having a wavelength at around 820 nm is added. For example, the first illumination light L1 with the wavelength at around 820 nm is generated by a combination of a xenon lamp and a bandpass filter having a center wavelength of 820 nm.

As an alternative to the above embodiment in which single first illumination light L1 is radiated onto the biological tissue S, a plurality of first illumination light L1 having different center wavelengths may be radiated onto the biological tissue S. For example, first illumination light L1 having a center wavelength of 545 nm and first illumination light L1 having a center wavelength of 584 nm may be radiated onto the biological tissue S.

Likewise, a plurality of second illumination light L2 having different center wavelengths may be radiated onto the biological tissue S. For example, a plurality of second illumination light L2 having center wavelengths selected from a range between 590 nm and 630 nm may be radiated onto the biological tissue S.

In this case, the plurality of first illumination light L1 are sequentially radiated onto the biological tissue S, so that a plurality of first images are acquired by the imaging unit 2. For the calculation of the indicator value I, the first image with the smaller pixel values may be used from the plurality of first images.

Furthermore, the plurality of second illumination light L2 are sequentially radiated onto the biological tissue S, so that a plurality of second images are acquired by the imaging unit 2. For the calculation of the indicator value I, the second image with the larger pixel values may be used from the plurality of second images.

According to this configuration, the indicator value I and the oxygen saturation can be calculated with higher accuracy.

As an alternative to the above embodiment in which the light source device 3 includes the LEDs 71, 72, 73, and 74 as white light sources, the white light sources may be light sources of another type. For example, the white light sources may be laser light sources, such as laser diodes (LDs), or white lamps, such as xenon lamps.

As an alternative to the above embodiment in which the illumination light L1 and L2 for acquiring the oxygen saturation image B are generated from the light Lg and Lr output from the white light sources 73 and 74, the light source device 3 may include dedicated light sources for the illumination light L1 and L2 separately from the white light sources 71, 72, 73, and 74.

As an alternative to the above embodiment in which the biological-image generating system 100 switches between the white-light-image mode and the oxygen-saturation-image mode based on an input from the user, the biological-image generating system 100 may automatically switch between the white-light-image mode and the oxygen-saturation-image mode at a predetermined timing. For example, the biological-image generating system 100 may alternately switch between the white-light-image mode and the oxygen-saturation-image mode so as to alternately acquire a white light image and an oxygen saturation image.

As an alternative to the above embodiment in which the biological-image generating system 100 is an endoscope system, the biological-image generating system may be a system of an arbitrary type that acquires an optical image of biological tissue. For example, the biological-image generating system may be a microscope system that includes an optical microscope for observing the inside of a biological organism.

### REFERENCE SIGNS LIST

2 imaging unit
3 light source device (light source unit)
4 image processing device (processor)
71, 72, 73, 74 LED, white light source
100 biological-image generating system
B oxygen saturation image (biological image)
L1 first illumination light
L2 second illumination light
S biological tissue

1. A biological-image generating method comprising:
acquiring a first image of biological tissue irradiated with first illumination light;
acquiring a second image of the biological tissue irradiated with second illumination light; and
generating a biological image from the first image and the second image,
wherein the first illumination light has a wavelength which is selected from a range between 500 nm and 600 nm and at which reflectance of the first illumination light is not dependent on an oxygen saturation,
wherein the second illumination light has a wavelength which is selected from a range between 600 nm and 800 nm and at which reflectance of the second illumination light is dependent on the oxygen saturation, and
wherein the generating the biological image comprises:
calculating an indicator value expressing the oxygen saturation from a first pixel value of each pixel in the first image and a second pixel value of each pixel in the second image; and
allocating a display mode corresponding to the indicator value to each pixel of the biological image.

2. The biological-image generating method according to claim 1,
wherein the wavelength of the first illumination light is selected from a range between 580 nm and 590 nm, and
wherein the wavelength of the second illumination light is selected from a range between 620 nm and 650 nm.

3. The biological-image generating method according to claim 1,
wherein the wavelength of the first illumination light is a wavelength at which an absorption coefficient of oxygenated hemoglobin and an absorption coefficient of reduced hemoglobin are equal to each other.

4. The biological-image generating method according to claim 3,
wherein a center wavelength of the first illumination light is selected from a wavelength at around 500 nm, a wavelength at around 525 nm, a wavelength at around 545 nm, a wavelength at around 575 nm, and a wavelength at around 584 nm.

5. The biological-image generating method according to claim 1,
wherein the indicator value is a ratio or a difference between the first pixel value and the second pixel value.

6. The biological-image generating method according to claim 5, further comprising:
determining a low oxygen region where the oxygen saturation is lower than or equal to a predetermined threshold value based on the indicator value.

7. The biological-image generating method according to claim 6,
wherein a display mode different from a display mode in another region of the biological image is allocated to the low oxygen region.

8. A biological-image generating system comprising:
a light source unit that outputs first illumination light and second illumination light;
an imaging unit that comprises an image sensor and that acquires a first image of biological tissue irradiated with the first illumination light and a second image of the biological tissue irradiated with the second illumination light; and
a processor configured to generate a biological image from the first image and the second image,
wherein the first illumination light has a wavelength which is selected from a range between 500 nm and 600 nm and at which reflectance of the first illumination light is not dependent on an oxygen saturation,
wherein the second illumination light has a wavelength which is selected from a range between 600 nm and 800 nm and at which reflectance of the second illumination light is dependent on the oxygen saturation, and
wherein the processor is configured to:
calculate an indicator value expressing the oxygen saturation from a first pixel value of each pixel in the first image and a second pixel value of each pixel in the second image; and
allocate a display mode corresponding to the indicator value to each pixel of the biological image.

9. The biological-image generating system according to claim 8,
wherein the wavelength of the first illumination light is selected from a range between 580 nm and 590 nm, and
wherein the wavelength of the second illumination light is selected from a range between 620 nm and 650 nm.

10. The biological-image generating system according to claim 8,
wherein the wavelength of the first illumination light is a wavelength at which an absorption coefficient of oxygenated hemoglobin and an absorption coefficient of reduced hemoglobin are equal to each other.

11. The biological-image generating system according to claim 10,
wherein a center wavelength of the first illumination light is selected from a wavelength at around 500 nm, a wavelength at around 525 nm, a wavelength at around 545 nm, a wavelength at around 575 nm, and a wavelength at around 584 nm.

12. The biological-image generating system according to claim 8,
wherein the processor is configured to calculate a ratio or a difference between the first pixel value and the second pixel value as the indicator value.

13. The biological-image generating system according to claim 12,
wherein the processor is configured to determine a low oxygen region where the oxygen saturation is lower than or equal to a predetermined threshold value based on the indicator value.

14. The biological-image generating system according to claim 13,
wherein the light source unit comprises a white light source for acquiring a white light image of the biological tissue, and
wherein the white light source outputs light containing the first illumination light and the second illumination light.
